(19) United States
(12) Patent Application Publication
(10) Pub. No.: US 2018/0078266 A1
(43) Pub. Date: Mar. 22, 2018

(54) ALIGNMENT APPARATUS

(71) Applicant: INLINE ORTHOPAEDICS PTY LTD, Samford, QLD (AU)

(72) Inventors: Don Fry, Samford (AU); David John Cooper, Samford (AU); David John Wadley, Samford (AU); Martin John Ford, Samford (AU)

(73) Assignee: INLINE ORTHOPAEDICS PTY LTD, Samford, QLD (AU)

(21) Appl. No.: 15/561,366

(22) PCT Filed: Mar. 24, 2016
(86) PCT No.: PCT/AU2016/050220

§ 371 Date: Sep. 25, 2017

(30) Foreign Application Priority Data
Mar. 25, 2015 (AU) 2015901083

(51) Int. Cl. A61B 17/17; A61B 34/20; A61B 90/00; A61B 34/00; A61F 2/46

(52) U.S. Cl. CPC A61B 17/1746; A61B 34/20; A61B 2017/00734; A61B 34/25; A61F 2/4657; A61B 90/37

(57) ABSTRACT

The present invention relates to an apparatus for aligning a sensor relative to at least two anatomical reference points of a patient's anatomy. In one embodiment, the apparatus includes: a body having a central axis; a sensor mount positioned relative to the body; at least two arms extending from the body, wherein two of said at least two arms are simultaneously and equidistantly moveable relative to the central axis; and at least two aligners connected to the at least two arms for aligning with said at least two anatomical reference points. The apparatus may also include an apparatus sensor. The present invention also relates to a surgical system for monitoring the orientation of a patient's anatomy, which includes the apparatus. Furthermore, the present invention also relates to a surgical system for guiding a surgical device to an optimal orientation relative to a patient's anatomy, wherein the surgical system includes the apparatus. The present invention also relates to: a method of aligning a sensor relative to at least two anatomical reference points of a patient's anatomy, and to a method of guiding a surgical device to an optimal orientation relative to a patient's anatomy. In one embodiment, the patient's anatomy is the pelvis.

### TECHNICAL FIELD

The present invention relates to an alignment apparatus for aligning a sensor relative to at least two anatomical reference points of a patient's anatomy, to surgical systems including the apparatus, and to methods of sensing the orientation of a patient's anatomy and of guiding a surgical device to an optimal orientation involving the apparatus.

### BACKGROUND ART

It will be clearly understood that, if a prior art publication is referred to herein, this reference does not constitute an admission that the publication forms part of the common general knowledge in the art in Australia or in any other country.

Correct alignment of a prosthetic component is very important in orthopaedic procedures, such as total hip replacement surgery. Optimal alignment may enhance the initial function and long term operability of the prosthetic component. Misalignment of the prosthetic component can result in patient pain and many other potential complications.

Correct alignment of a prosthetic component can be complex, involving multiple steps. Total hip replacement surgery, for example, typically involves: severing the femoral head and dislocating the head of the femur from the acetabulum; reaming the acetabulum to fit an acetabular cup and then inserting the acetabular cup; shaping the femoral canal, and then fitting a prosthetic femoral component into the canal; and fitting the prosthetic femoral component to the acetabular cup. Consequently, to successfully mimic a normal hip joint the acetabular cup and the prosthetic femoral component must be correctly aligned with each other and with the patient's pelvis and femur.

One of the most difficult steps in total hip replacement surgery is correctly aligning the acetabular cup in the acetabulum. For example, in a study spanning 4226 hip replacement surgeries in multiple centres, surgeons were only able to align the acetabular component within an optimal zone in less than 30% of procedures (Langton, D. J. et al. (2011), J Bone Joint Surg[Br] 93-B:164-71). This optimal zone corresponds to a safe zone derived from an earlier work based upon an analysis of serum metal ion results and explants and was defined as 40° to 50° inclination and 10° to 20° anteversion. Consequently, it was concluded that “there is overwhelming evidence to show that surgeons cannot consistently position the acetabular components precisely. Without exception, studies show wide variations in the angles of inclination of the acetabular component and, to an even greater extent, its anteversion.” This is significant as an error in acetabular cup placement of as little as 5° can result in patient complications.

In total hip replacement surgery misalignment of the acetabular cup on the pelvis can result in dislocation of the hip joint, misalignment of the patient's leg, incorrect leg length, decreased joint motion and joint pain. A misaligned acetabular cup on the pelvis may affect the patient's posture, the biomechanics of the lower limbs (for example affecting the range of movement of the hip), and the degree of curvature of the thoracic and cervical spines. The long term effects of a misaligned acetabular cup can include accelerated wear of the components, aseptic loosening of the components and potentially early revision surgery. Furthermore, misalignment may increase the leaching of the prosthesis metal components (for example into the blood stream), which can lead to immune system problems.

To illustrate the scale of this problem, it has been estimated that 959,000 primary and revision total hip replacement procedures were undertaken per year in the United States, Spain, Portugal, The Netherlands, Canada, France, Italy Switzerland and Germany (Kurtz, S. M. (2010) Paper #365. Presented at the 56th Annual Meeting of the Orthopaedic Research Society. Mar. 6-9, 2010. New Orleans). The number of procedures is only expected to increase as an increasingly aging population is driving growth in the orthopaedics market worldwide. Furthermore, hip prostheses at present last on average 12 to 15 years even though manufacturers' laboratory test conditions indicate they should last 30 years. In the United States alone, it has been estimated that the cost of revision surgery for total hip replacement procedures exceeds US$1 billion per annum (Katz, J. N. (2007) The Orthopaedic Journal at Harvard Medical School 9:101-106).

In an effort to accurately align a surgical device (such as an acetabular cup inserter) with the acetabulum, surgeons have been known to use the position of anatomical landmarks around the acetabulum as a guide. However, these landmarks may be obscured during surgery, making it very difficult for a surgeon to assess the optimal orientation.

Another approach is to use a sensor in conjunction with a surgical device to sense the orientation of the surgical device. However, to accurately orient the surgical device with reference to the acetabulum, surgeons need to know the orientation of the acetabulum. One method may involve use of the sensed orientation of the acetabular rim for aligning the surgical device. However, a problem with this approach is that it takes time during an operation to sense the orientation of the acetabular rim, and it is better for elderly patients in particular (for whom hip replacement operations are more common) to be under anaesthetic for shorter periods of time. Furthermore, there may be natural variations in the surface of the acetabular rim which may introduce errors.

A further approach is to sense the orientation of the pelvis as a whole and to use this information to determine the correct orientation for a surgical device. However, a difficulty with this approach is that the size, shape and contour of pelvises vary between patients and consequently it can be difficult to accurately sense the orientation of the pelvis as a whole.

The present invention is directed to, inter alia, an apparatus for aligning a sensor relative to a patient's anatomy, such as the pelvis, and which may at least partially overcome at least one of the abovementioned disadvantages or provide the consumer with a useful or commercial choice.

### SUMMARY OF INVENTION

With the foregoing in view, the present invention in one form, resides broadly in an apparatus for aligning a sensor relative to at least two anatomical reference points of a patient's anatomy.

In a first aspect, the present invention provides an apparatus for aligning a sensor relative to at least two anatomical reference points of a patient's anatomy, wherein the apparatus includes:
a. a body having a central axis;
b. a sensor mount positioned relative to the body;
c. at least two arms extending from the body, wherein two of said at least two arms are simultaneously and equidistantly moveable relative to the central axis; and
d. at least two aligners connected to the at least two arms for aligning with said at least two anatomical reference points.

Advantageously, the apparatus includes two arms which extend from the body and which are simultaneously and equidistantly moveable relative to the central axis. Consequently, the central axis is positionable in a defined orientation relative to the patient's anatomy, regardless of the distance between the two anatomical reference points to be aligned with the aligners connected to the two arms. This permits more precise measurement of the orientation of the patient's anatomy.

Each arm may extend from the body in any orientation. In one embodiment, the two of said at least two arms (hereinafter “the two arms”) extend on opposite sides of the body or central axis; and especially in opposite directions to each other. The two arms may extend perpendicularly to the central axis. In one embodiment, the two arms (and especially all of the at least two arms) extend in a substantially common plane.

The two arms may be simultaneously and equidistantly moveable relative to the central axis in any suitable way. In one embodiment, the apparatus includes an arm mechanism for simultaneously and equidistantly moving the two arms relative to the central axis. The arm mechanism may operate manually or electronically, especially manually. The arm mechanism may be geared. A geared mechanism may be advantageous as this permits incremental movement of the two arms allowing more precise alignment with the patient's anatomy. The arm mechanism may be a rack and pinion mechanism. Therefore, in one embodiment the arm mechanism may include one pinion and the two arms may each include a rack (which may be teeth on the two arms). In this embodiment, as the pinion is rotated the teeth on the pinion engage with teeth on the two arms which simultaneously and equidistantly moves the arms relative to the central axis. The pinion may further include a handle for actuating the pinion.

The arm mechanism may include a brake to prevent movement of the two arms. The brake may include a brake pad. In one embodiment, when the mechanism is a rack and pinion mechanism the brake may be connected to or be integral with the pinion. In this embodiment, the brake may include a brake pad and a knob, wherein rotation of the knob drives the pinion handle into the brake pad, preventing the handle from turning to actuate the pinion.

In some embodiments, the apparatus includes from 2 to 6 arms, especially from two to 5 arms, more especially from 2 to 4 arms, most especially 3 arms. When the apparatus includes three arms, the arms in the apparatus may generally extend relative to each other in a “Y”-shape or a “T”-shape. Each said arm may be curved or substantially straight, especially substantially straight. Each said arm may be moveable to enable each aligner connected to each arm to be located in alignment with each anatomical reference point.

The body may include at least one sleeve. Each sleeve may support at least one arm. At least one arm (or each arm) may extend through each sleeve. A first end of each sleeve may be open or partially open for accommodating at least one arm. A second end of each sleeve may be open, partially open, or closed (in which case the arm would not extend through the sleeve). Each sleeve is especially open or partially open at both ends. Each sleeve may be substantially cylindrical, rectangular or cuboid in shape (especially rectangular).

A first sleeve may be for the two arms. A second sleeve may be for a third arm. A third sleeve may be for a fourth arm, a fourth sleeve may be for a fifth arm or a fifth sleeve may be for a sixth arm. When the body includes two or more sleeves, each said sleeve may be in any suitable orientation relative to each other sleeve. In one embodiment, the body includes two sleeves, and the sleeves may especially be oriented substantially perpendicular to each other. The first and second sleeves may be oriented such that the two arms extend substantially perpendicularly to the third arm.

The apparatus may further include at least one lock for locking the at least one arm in position. Each lock may be of any suitable form. In one embodiment, the at least one lock includes a fastener. Rotational movement of the fastener may lock the arm in position (typically at a point where the aligner connected to the arm is in alignment with an anatomical reference point). The fastener may traverse two opposed walls of a sleeve. Rotational movement of the fastener may draw the walls of the sleeve together, thereby locking the arm in position. The arm may include a longitudinally extending slot, through which the fastener extends. Alternatively, the fastener may bear on the arm to thereby lock the arm in position.

The apparatus may include a third arm. For avoidance of doubt, the “third arm” is one of said at least two arms (and may be as described for said at least two arms). The apparatus may include a third arm extending from the body, and a third aligner connected to the third arm for aligning with a third anatomical reference point, wherein said third arm is moveable relative to the body. For avoidance of doubt, the “third aligner” is one of said at least two aligners (and may be as described for said at least two aligners). The third arm may extend along the central axis. The body may include a second sleeve for the third arm. The second sleeve may be substantially rectangular in shape. The apparatus may include a lock for the third arm. The lock may include a fastener which traverses the second sleeve. Rotational movement of the fastener may draw opposed walls of the second sleeve together, thereby locking the third arm in position. The third arm may include a longitudinally extending slot, through which the fastener extends. Alternatively, the fastener may bear on the arm to thereby lock the arm in position.

The sensor mount may be located or be positionable on the body, or on one of the arms (especially the third arm). The sensor mount may be releasably attached to the body or one of the arms (especially the third arm). In one embodiment, the sensor mount may be clampable to one of the arms (especially the third arm). The clamp may include a channel for accommodating a portion of one of the arms (especially the third arm). The clamp may also include a fastener which traverses the channel (especially wherein the channel includes two opposed walls and the fastener traverses the two opposed walls). Rotation of the fastener may draw the walls of the channel together to clamp the sensor mount on the body or on one of the arms (especially the third arm). In one embodiment, the third arm includes a longitudinally extending slot and the clamp fastener extends through the slot.

The apparatus may further include a sensor (hereinafter “apparatus sensor”). The sensor mount may include the apparatus sensor, or the apparatus sensor may be releasably mounted to the sensor mount. The apparatus sensor may be releasably mounted to the sensor mount by any suitable mechanism, such as by friction-fit, interference-fit, tongue in groove, bayonet coupling, via a hook and loop fastener (such as Velcro™) or the like. In one embodiment, the sensor mount includes a docking port for mounting the apparatus sensor. The apparatus sensor may slideably engage with the docking port, or the apparatus sensor may be screwed into the docking port. The sensor mount may also include a clamp to secure the apparatus sensor on the sensor mount.

The orientation sensed by the apparatus sensor may be one or more of the pitch, roll and yaw of the patient's anatomy; especially two or more of pitch, roll and yaw; especially all of pitch, roll and yaw. Pitch and roll are sensed relative to the horizontal, and yaw is sensed relative to the patient axial line as determined by the surgeon. In hip surgery, for example, yaw may be sensed relative to the patient sagittal plane, which is the longitudinal planar axis, passing through the head, spine and pelvis of the body, bisecting the body into two equal parts.

The apparatus sensor may comprise at least one sensor for sensing the orientation of the patient's anatomy. The at least one sensor senses at least one of pitch, roll and yaw of the patient's anatomy; especially at least two of pitch, roll and yaw; and most especially all of pitch, roll and yaw. In one embodiment, the at least one sensor is selected from one or more of a gyroscope, a magnetometer, an accelerometer, an inclinometer and an inertial sensor. Exemplary sensors/sensor combinations are available from Xsens (http://www.xsens.com). In one embodiment, the sensor is or includes an accelerometer supported magnetometer. The sensor may also be or include an atomic gyroscope. In another embodiment, the sensor may act in combination with laser pointing, or a beacon located in proximity to the sensor (for example in the operating theatre), and this embodiment may be especially advantageous when sensing yaw. The apparatus sensor may be a 3 axis self-powered Wi-Fi transmitter.

The apparatus sensor may be connected to a power supply. For example, in use the apparatus sensor may be connected to an external power supply such as a wall socket. Alternatively, the apparatus sensor may comprise a power supply, such as a battery. Any suitable battery may be used. The battery may be rechargeable or not rechargeable. For example, the battery may be rechargeable by coupling the sensor to a power cord or by inductive charging. A battery rechargeable by inductive charging may be especially advantageous if the apparatus sensor is sterilisable. The battery may be replaceable and/or sterilisable. In a further embodiment, the apparatus sensor may be disposable in which case the battery may not be replaceable. The power supply may be, for example, a sterilisable lithium ion battery. One, two or more of such batteries may be used. The apparatus sensor may also comprise an on/off switch for activating the sensor. The apparatus sensor may also include an indicator (such as a light) for indicating the remaining power in the power supply.

If the apparatus sensor is sterilisable, then it may include an insulator for insulating the electronic components from chemical, thermal or pressure effects. The insulator may protect these components against standard autoclave sterilising or gas approved sterilisation. In one embodiment, the insulator is a casing. In one embodiment, the apparatus sensor is sterilisable. However, a non-sterile apparatus sensor may be used if it is inserted inside a sterile housing.

The apparatus may include at least one aligner connected to each arm; especially one aligner for each arm. In one embodiment, the at least two arms are oriented in substantially common plane, and the at least two aligners are oriented transversely (especially substantially perpendicularly or perpendicularly) to the common plane. The at least two aligners may extend substantially parallel (or parallel) to each other. Each aligner may be moveable relative to each arm. In one embodiment, each arm is moveable in a transverse (especially substantially perpendicular or perpendicular) direction to the direction of movement for each arm (or to the substantially common plane).

Each arm may terminate at a sleeve, and each aligner may be moveable within said sleeve. In one embodiment, each aligner includes a rod, and each rod is moveable within each arm sleeve. Each aligner may be lockable in position, and this may be achieved in any suitable way. For example, the aligner may define a plurality of apertures or detents which are engageable with a fastener (such as a screw) mounted to each arm sleeve. Each aligner may be the same length.

Each aligner may include an end for alignment with each anatomical reference point. Each end may be of any suitable form or shape. For example, each aligner may have a bulbous or tapered end. Each end may be blunt or include a needle (which may be advantageous for precise alignment). Each end may include a contoured terminal surface shaped to conform with an anatomical reference point. For example, each end may include a groove or ridge, especially for engagement with a bony surface (for example, an aligner end intended to align with an iliac spine may include a groove. An aligner end intended to align with at least one pubic crest may include a centrally protruding ridge to enable establishment of the median sagittal plane). Each end may be removable and/or replaceable. Each end may terminate with a pad. The pad may be, for example of 10 to 20 mm diameter. Each end may be selected based on the requirements of individual patients. For example, large amounts of material (e.g. tissue) between the skin and the bone of an anatomical reference point may obscure the anatomical reference point (this may occur in obese patients for example) which may increase error. To ameliorate this error, ends of smaller diameter may be used (and needles may be particularly advantageous).

In use, each aligner may especially be positioned so that they are all the same length from the at least two arms. If a patient is obese or overweight it may be advantageous for the aligner ends to be further away from the arms so that the patient's abdomen may be accommodated. However, to improve accuracy it is typically advantageous for the distance between the aligner ends and the arms to be as short as possible.

In one embodiment, the patient's anatomy is selected from the group consisting of: the pelvis, the scapula, the femur, the tibia, the medial malleolus, the radius, the ulna, the humerus, the tibia and the fibula; especially the pelvis or the scapula; most especially the pelvis. Therefore, the apparatus may be used in orthopaedic surgical procedures, such as in surgery on the joint of a patient, especially joint reconstruction, and most especially total joint replacement. The joint may be selected from the group consisting of: a hip, a shoulder, a knee, an ankle, a finger, a thumb, a toe (especially the first metatarsophalangeal (MTP) joint or the big toe), an elbow, and a wrist; especially a hip or a shoulder; most especially a hip. The apparatus may be used in, for example, total hip replacement surgery, total knee arthroplasty, high tibial osteotomy, total shoulder replacement surgery, total wrist replacement surgery, total ankle replacement surgery, surgery to orient the thumb, fingers or toes or total elbow replacement surgery. In a further embodiment, the surgical system may be for use in orthopaedic resurfacing, such as in hip resurfacing.

In one embodiment, the anatomical reference points are points that are visible to a surgeon through the skin (i.e. without operation). When the patient's anatomy is the pelvis, the anatomical reference points may be selected from the group consisting of: the iliac crest, the pubic crest (or pubic symphysis) and the sacrum. The iliac crest terminates anterosuperiorly at the anterior superior iliac spine (ASIS) and posterosuperiorly at the posterior superior iliac spine (PSIS). Therefore, the anatomical reference points may be selected from the group consisting of one or two ASIS, one or two PSIS, at least one pubic crest and the sacrum. The two arms especially may be for registering with the iliac crests, especially the two ASISs or the two PSISs. The third arm may be for registering with the at least one pubic crest or the sacrum. The “at least one pubic crest” may include one or both pubic crests, as well as the pubic symphysis which lies between the two pubic crests. The two pubic crests are in close proximity, and accordingly one aligner may be suitable for aligning with one or both pubic crests.

The apparatus may be for registering with any number of anatomical reference points. In one embodiment, the apparatus is for registering with from 2 to 6 anatomical reference points; especially from 2 to 4 anatomical reference points; more especially 3 anatomical reference points. When the patient's anatomy is the pelvis, the anatomical reference points may be: (i) two anterior superior iliac spines, and the at least one pubic crest; or (ii) two posterior superior iliac spines and the sacrum.

The apparatus may be for defining the patient's coronal plane (or especially pelvic plane). The apparatus sensor may also be positionable on or relative to the patient's median sagittal plane. The apparatus may be for defining the patient's sagittal (or median sagittal) plane.

The apparatus (or the various components of the apparatus) may be made of any suitable materials. In one embodiment, one or more of the body, sensor mount, arm mechanism, arms, aligner rods and at least one lock may be made of a non-magnetisable metal, especially steel (especially stainless steel) or titanium. Titanium may be used for the aligner rods and stainless steel for the body, sensor mount, arm mechanism, arms, and the at least one lock. In another embodiment, the aligner ends may be made of a polymer, such as a heat resistant polymer (for example, heat resistant nylon). The apparatus (or various components of the apparatus) may be sterilisable.

In a second aspect, the present invention relates to a method of aligning the apparatus of the first aspect of the present invention relative to a patient's anatomy, or to at least two anatomical reference points of a patient's anatomy. The method may include actuating the arm mechanism to simultaneously and equidistantly move the two arms relative to the central axis to thereby align the aligners connected to the two arms with two anatomical reference points. The method may also include activating the brake to prevent movement of the two arms. The method may further include moving the third arm along the central axis to thereby align the aligner connected to the third arm with an anatomical reference point. The method may further include activating the lock to lock the third arm in position. The method may also include releasably attaching the sensor mount to at least one arm, or to the body. The method may also include releasably mounting the apparatus sensor to the sensor mount.

When aligning the apparatus of the first aspect of the present invention relative to the patient's anatomy, the patient may be positioned on their side. When the patient's anatomy is the pelvis, positioning the patient vertically on their side may be effective if the patient's abdomen is not large, but for patients with a large abdomen (for example obese patients) it may be advantageous to position the patient off the vertical, for example 20° off vertical (as this may lessen the bulge on the patient's lower iliac spine). The apparatus may also be effectively used with the patient lying on their back.

In one embodiment of the second aspect, the present invention relates to a method of aligning a sensor relative to at least two anatomical reference points of a patient's anatomy, wherein the method includes the steps of:
a. simultaneously and equidistantly moving the two of said at least two arms of the apparatus of the first aspect relative to the central axis; and
b. aligning the at least two aligners with said at least two anatomical reference points.

In a third aspect, the present invention relates to a method of sensing the orientation of a patient's anatomy. The method may include the steps of the second aspect. The method may also include the step of activating the apparatus sensor to thereby sense the orientation of the patient's anatomy (or to thereby define the patient's coronal plane (or especially pelvic plane)).

Features of the second and third aspects of the present invention may be as defined for the first aspect.

In a fourth aspect, the present invention relates to a surgical system for monitoring the orientation of a patient's anatomy, wherein the system includes:
a. an apparatus for aligning an apparatus sensor relative to at least two anatomical reference points of a patient's anatomy to thereby sense an initial orientation of the patient's anatomy, wherein the apparatus includes:
   i. a body;
   ii. the apparatus sensor positioned relative to the body;
   iii. at least two arms extending from the body; and
   iv. at least two aligners connected to the at least two arms for aligning the apparatus with said at least two anatomical reference points; and
b. a patient sensor mountable relative to the patient's anatomy, wherein the patient sensor is for sensing changes in the orientation of the patient's anatomy;
wherein the surgical system is configured to monitor the orientation of the patient's anatomy by combining the initial orientation sensed by the apparatus sensor with the changes in orientation sensed by the patient sensor.

The apparatus defined in the fourth aspect may include a sensor mount. The apparatus defined in the fourth aspect may include a body having a central axis. Two of the at least two arms of the apparatus may also be simultaneously and equidistantly moveable relative to the central axis. Features of the apparatus, patient's anatomy and anatomical reference points defined in the fourth aspect of the present invention may be as defined in the first aspect.

The patient sensor may have the same features as the apparatus sensor defined above.

The patient sensor may be mountable relative to the patient's anatomy in any suitable way. In one embodiment, the patient sensor may be mounted to the patient's anatomy, for example by way of a fastener such as pedicle screws or an adhesive. For example, the adhesive may be an adhesive tape, such as Elastoplast®, for taping the patient sensor to or over the patient's anatomy. The patient sensor may also be mounted relative to the patient's anatomy, for example by way of a patient sensor support. Therefore, the surgical system may include a patient sensor support.

The patient sensor support may take any suitable form. In one embodiment, the patient sensor support includes a body. The body may be of any suitable shape. Advantageously, when using the alignment apparatus it is not necessary to precisely align the patient sensor on the patient's anatomy. Consequently, the patient sensor support may be relatively small and typically need only be large enough to mount the patient sensor and to be mounted to the patient. Therefore, the patient sensor support body may have a substantially square shape or a substantially rectangular shape. The patient sensor support may be mounted on any suitable part of a patient's anatomy, but a preferred location is where there is minimal tissue between the bone and the skin (to minimise unnecessary movement of the patient sensor). An exemplary location when performing operations on the pelvis is the sacrum.

The body of the patient sensor support may include a patient sensor mount for mounting the patient sensor. The patient sensor may be releasably mounted to the patient sensor mount by any suitable mechanism, such as by friction-fit, interference-fit, tongue in groove, bayonet coupling, via a hook and loop fastener (such as Velcro™) or the like. In one embodiment, the patient sensor mount includes a docking port for mounting the patient sensor. The patient sensor may slideably engage with the docking port, or the patient sensor may be screwed into the docking port. The patient sensor mount may also include a clamp to secure the patient sensor. In another embodiment, the patient sensor and patient sensor mount may be permanently coupled together.

The patient sensor support may include at least one fastener for mounting the patient sensor relative to the patient's anatomy. The fastener may include an adhesive for adhering the body to the patient's skin (the patient's skin may lie over the patient's anatomy). The fastener may be an adhesive tab, for example an electrocardiography (ECG) electrode tab. The fastener may also be strapping or adhesive tape for holding the body against the patient's anatomy. The fastener may be pedicle screws for screwing the body onto a patient's anatomy. The fastener may also be suction caps to secure the body to or over the patient's anatomy. The fastener may also comprise a hook and loop fastener, especially a circular hook and loop fastener. An example hook and loop fastener is Velcro™. Combinations of two or more of the above fasteners may also be used.

The patient sensor support may include any suitable number of fasteners, especially from 1 to 6 fasteners, more especially from 2 to 6 fasteners, or from 2 to 4 fasteners, especially 4 fasteners.

The patient sensor support may include at least one clamp for clamping the at least one fastener to the body. In an exemplary embodiment, each at least one fastener may be an adhesive tab. The adhesive tab may include an adhesive surface for adhering to the patient's skin. The adhesive tab may also include a projection (which may be substantially cylindrically shaped) opposite the adhesive surface. Exemplary adhesive tabs are those used in electrocardiography (ECG). The clamp may clamp the projection of the adhesive tab.

The clamp may be of any suitable type. In one embodiment, the clamp is pivotally mounted to the body and moveable between an open and a closed position. The clamp may include a biasing member (such as a spring) to bias the clamp to the closed position. The clamp may include a lever. The lever may include a jaw at one end and an actuator at the opposite end. The jaw may be for clamping the fastener against the body. The jaw may be of any suitable shape, but in an exemplary embodiment may include a U-shaped slot (especially for clamping the projection of an adhesive tab). The actuator may be depressed by an operator to move the clamp to an open position. The patient sensor support may include four clamps mounted to the body.

The patient sensor support may be flexible or substantially rigid, especially substantially rigid. The body of the patient sensor may be made of any suitable material, especially a plastic such as polytetrafluoroethylene (PTFE) or polycarbonate. The base may be especially made of an X-ray translucent material. The patient sensor support may be sterilisable.

The surgical system may include at least one patient sensor. In one embodiment, the surgical system includes only one patient sensor, but two may be used. Using more than one patient sensor may provide redundancy in case one patient sensor is accidentally displaced from the patient's anatomy during the operation. If the surgical system comprises more than one patient sensor, then the patient sensors may be identical to each other or different.

In a fifth aspect, the present invention relates to a method of monitoring the orientation of a patient's anatomy. The method may include using the system of the fourth aspect of the present invention. The method may include the steps of the second aspect. The method may also include the step of mounting the patient sensor relative to the patient's anatomy. For example, this step may include one or more of: releasably mounting the patient sensor to the patient sensor mount; clamping the at least one fastener to the patient sensor support body; and fastening the at least one fastener to the patient. In one embodiment, first the at least one fastener is clamped to the patient sensor support body; second the at least one fastener is fastened to the patient; and third the patient sensor is releasably mounted to the patient sensor mount. The method may also include the step of aligning the apparatus relative to at least two anatomical reference points on the patient's anatomy. The method may also include the step of releasably mounting the apparatus sensor to the apparatus sensor mount. The method may further include the step of activating the patient sensor and the apparatus sensor to thereby sense the orientation of the patient's anatomy.

The orientation of the patient's anatomy as measured by the apparatus sensor provides an initial orientation. The initial orientation is typically more precise and may be used to define the patient's coronal plane (or especially pelvic plane), and possibly also the patient's median sagittal plane. A pre-operative scan may be performed, for example by X-ray, to ascertain the location and orientation of features of the patient's anatomy relative to the anatomical reference points (or relative to the coronal plane (or especially pelvic plane)). Therefore, if the patient's anatomy is the pelvis, then a pre-operative X-ray (for example) may be used to define the position and orientation of the acetabulum relative to anatomical landmarks such as the two anterior superior iliac spines (ASIS), the two posterior superior iliac spines (PSIS), the pubic crests (and pubic symphysis) and the sacrum. It may be advantageous to define the coronal and sagittal planes using the apparatus sensor, as the inclination and anteversion of the acetabulum are typically measured with reference to these planes.

If the patient sensor and apparatus sensor are active at the same time, then the patient sensor may be paired with the apparatus sensor (for example directly, via a relay sensor, or via a monitor which acquires data from the patient and apparatus sensors independently). The orientation of the patient's anatomy as measured by the patient sensor senses changes in the orientation of the patient's anatomy, and this data may be used to update the initial orientation sensed by the apparatus sensor. Consequently, after the apparatus and patient sensors are paired, the apparatus may be removed from the patient as it is no longer needed.

In a sixth aspect, the present invention provides a surgical system for guiding a surgical device to an optimal orientation relative to a patient's anatomy, wherein the surgical system includes:
a. the surgical system for monitoring the orientation of a patient's anatomy of the fourth aspect of the present invention;
b. an orientation sensor for sensing the orientation of the surgical device; and
c. a monitor for monitoring the orientation of the surgical device relative to the monitored orientation of the patient's anatomy, and for guiding the surgical device to an optimal orientation relative to the monitored patient's anatomy.

The surgical device may be or include a surgical implement, such as a reamer. The reamer may be an acetabular or glenoid cavity reamer (especially acetabular reamer). In one embodiment, the surgical device may also be or include a prosthetic component, such as a prosthetic component for hip or shoulder replacement surgery, especially an acetabular or humeral cup (especially an acetabular cup). The acetabular cup may be for receiving a femur or a prosthetic femur. In this embodiment, the surgical device may also include a placement device for placing the prosthetic component. In a further embodiment, the surgical device may be a jig, especially for operating on a knee. The surgical system may include the surgical device.

The surgical device may include the orientation sensor. Alternatively, the orientation sensor may be releasably mounted and/or mounted relative to the surgical device. The system may further include an orientation sensor mount for releasably mounting the orientation sensor to the surgical device. The orientation sensor may be releasably mounted to the orientation sensor mount by any suitable mechanism, including those described above for releasably mounting the apparatus sensor to the apparatus sensor mount.

The orientation sensor mount may include one or more of a shock absorber (especially for absorbing shocks associated with driving a prosthetic component into the required position), a clamp for clamping the orientation sensor mount to the surgical device, and a spacer for spacing the orientation sensor from the clamp. The clamp may include a fastener. An advantage of using a spacer is that when the system is used, the orientation sensor may be offset and be less likely to obscure a surgeon's line of sight down the central axis of the surgical device. In one embodiment, the orientation sensor sensing portion is offset from the longitudinal axis of the surgical device.

Any suitable shock absorber may be used, and the shock absorber may absorb shocks in one, two or more directions, especially in one or two directions. The shock absorber may, for example, comprise a biasing member (such as a spring), and/or comprise pneumatic absorption (for example, like a piston). Similarly, any suitable fastener may be used, including bolts and lock nuts. It may not be necessary for the orientation sensor to include a shock absorber when the orientation sensor is attached to an acetabular reamer, as the effect of the acceleration of the acetabular reamer on the orientation sensor may be effectively ameliorated electronically.

In a further embodiment, the orientation sensor mount may include a cradle or bracket for holding a monitor (as discussed further below). Such a cradle or bracket may be designed to absorb shock, for example similar to those designed for motorcycle GPS. Alternatively, the system may include a stand for holding a monitor. The stand may be independent to the orientation sensor mount. The stand may be positionable on the floor of the operating theatre, especially in front of the surgeon.

The orientation sensor may have the same features as the apparatus sensor defined above. In one embodiment, the orientation sensor may be powered by the surgical device (such as when the surgical device is an acetabular reamer).

The orientation sensor mount may be made of any suitable material, including for example of a sterilisable material, such as metal or plastic, especially metal, more especially stainless steel. The orientation sensor mount may be made of a non-magnetisable material. The orientation sensor mount may be of any suitable shape and may be flexible or substantially rigid. The orientation sensor mount may be sterilisable.

The monitor monitors the orientation of the surgical device relative to the monitored patient's anatomy in real time. The monitor may monitor at least one of (and especially all of) the pitch, roll and yaw of the surgical device relative to the patient's anatomy. The monitor may store or data log information from the surgery. This information may indicate how many operations have been performed using the system; and/or may provide a full or partial record of the monitored orientation of the patient's anatomy, the sensed orientation of the surgical device, and/or the orientation of the surgical device relative to the patient's anatomy. The information may be transferable from the monitor to another medium, such as to a memory card. In one embodiment, the monitor comprises a data processor, a computer-readable storage medium, a microprocessor and/or a central processing unit (CPU). The monitor need not be a single piece of equipment. The monitor may be sterilisable.

The monitor may also communicate (especially to a surgeon) one or more of the monitored orientation of the patient's anatomy, the sensed orientation of the surgical device and the monitored orientation of the surgical device relative to the patient's anatomy.

The monitor may further comprise a communicator for communicating (especially to a surgeon) one or more of, especially all of, the monitored orientation of the patient's anatomy, the sensed orientation of the surgical device, and the orientation of the surgical device relative to the patient's anatomy. The communicator may visually communicate. For example the communicator may include a visual display such as a computer screen. The visual display may provide a graphical or numerical illustration of the orientation of the surgical device relative to the patient's anatomy and the optimal orientation for the surgical device relative to the patient's anatomy. The communicator may also audibly communicate (for example via an audio speaker).

The optimal orientation of the surgical device relative to the patient's anatomy may be calculated in any suitable way. In one embodiment, the patient and apparatus sensors are used to monitor the orientation of the patient's anatomy, and this together with pre-operative information (such as X-rays as discussed above) may be used to calculate the optimal orientation. For example, if the patient's anatomy is the pelvis the apparatus may be used to establish the orientation of the pelvis, and this information together with a pre-operative X-ray may be used to determine the orientation of the acetabulum which in turn allows determination of the optimal orientation of a surgical device for the acetabulum. When calculating the optimal orientation, the monitor may consider natural variations in the patient's anatomy as determined by a pre-operative measurement (such as an X-ray). An exemplary natural variation is a pelvic tilt angle, which is the difference between the vertical and a line drawn between the centre of rotation of each of the patient's hips, when the patient is positioned on an operating table with one hip vertically above the other, i.e. lying on their side. The optimal orientation may comprise at least one of (and especially all of) the pitch, roll and yaw of the surgical device relative to the patient's anatomy.

The monitor may guide the surgical device to an optimal orientation by communicating the difference between the optimal orientation of the surgical device and the orientation of the surgical device relative to the monitored patient's anatomy. As described above, the communicator may communicate via visual or audio signals (where, for example, the pitch, tone or duration of the audio signal guides the surgeon). In an exemplary embodiment, the visual signal may be a visual display including a horizontal and a vertical axis which bisect (illustrating roll and pitch), wherein the point at which the axes cross is the optimal pitch and roll for the surgical device relative to the patient's anatomy, and a dot on the display illustrates the surgical device's current orientation. The graphical display may also include a third axis which illustrates yaw, wherein the centre of the axis is the optimal yaw for the surgical device relative to the patient's anatomy and a marked position on the axis illustrates the current yaw.

The patient sensor or the orientation sensor may be co-located with the monitor. Advantageously, this may allow a surgeon to better see a read-out from the communicator during the operation. Alternatively, the monitor may be remote to the orientation sensor and the patient sensor. An advantage of a remote monitor is that the monitor may not require sterilisation between operations which could potentially damage some of the electronic components in the monitor. In alternative embodiments, the monitor and/or the communicator are sterilisable or disposable.

The monitor may be, for example, a computer (including devices such as laptops, tablets, smartphones, PDAs, iPads®, iPhones® and iPods®).

The surgical system may include at least a second communicator. The at least a second communicator may be positioned remote to the operation (for example on a wall of the theatre).

The apparatus, patient and orientation sensors, and the monitor, may all be in communication. The sensors may be in communication with each other, or via the monitor. The communication may be one-way or two-way, especially two-way. The communication may be via a cable or be wireless (such as via a wireless protocol for exchanging data over a short distance personal area network including Bluetooth™ or Wi-Fi). The communication should be non-invasive so as to avoid interfering with other electronic equipment in the operating theatre. The communication may be at a frequency and bandwidth that does not interfere with other hospital equipment. Furthermore, the wattage of the components may be configured to be allowable in operating theatres.

In another embodiment, the surgical system further comprises a tray for calibrating the apparatus, patient and/or orientation sensors, especially such that the sensors are parallel to each other. The sensors may be calibrated when in the tray, such that the pitch, roll and/or yaw for the sensors are set to zero. In the tray the sensors may be positioned so that they are generally uniformly aligned.

In a seventh aspect, the present invention provides a method of guiding a surgical device to an optimal orientation relative to a patient's anatomy. The method may include the steps of the method of the fifth aspect of the present invention. In addition, the method may include the step of releasably mounting the orientation sensor to the orientation sensor mount. The method may also include activating the orientation sensor. The method may further include activating the monitor. The method may further include the steps of: determining the optimal orientation of the surgical device, and/or monitoring the orientation of the surgical device relative to the monitored patient's anatomy. Features of the seventh aspect may be as described for the sixth aspect.

In variations of the above preceding description, the apparatus sensor may be used as the patient sensor, or the apparatus sensor may be used as the orientation sensor. To use the apparatus sensor as the patient sensor, the apparatus sensor is used in the apparatus to sense the initial orientation of the patient's anatomy. Then, with the orientation sensor remaining stationary, the apparatus sensor is paired to the orientation sensor and then the apparatus sensor is transferred to be used as the patient sensor.

Alternatively, the use the apparatus sensor as the orientation sensor, the apparatus sensor is used in the apparatus to sense the initial orientation of the patient's anatomy. Then the apparatus sensor is paired to the patient sensor (which is mounted to the patient's anatomy) and then the apparatus sensor is transferred to be used as the orientation sensor. While this approach uses fewer sensors, it may result in difficulties due to sterilisation. For example, after a sterilised apparatus has contacted the patient the apparatus is no longer considered sterile and therefore the non-sterile apparatus sensor may not be permitted to be transferred to a sterile surgical device for use as the orientation sensor.

In one embodiment of the seventh aspect, the present invention provides a method of guiding a surgical device to an optimal orientation relative to a patient's anatomy, wherein the method includes the steps of:
a. simultaneously and equidistantly moving the two of said at least two arms of the apparatus of the first aspect relative to the central axis;
b. aligning the at least two aligners with at least two anatomical reference points of the patient's anatomy;
c. sensing the orientation of the patient's anatomy with the apparatus sensor;
d. sensing the orientation of the patient's anatomy with a patient sensor mounted relative to the patient's anatomy, wherein the patient sensor is for sensing changes in the orientation of the patient's anatomy;
e. pairing the apparatus sensor with the patient sensor, to thereby monitor the orientation of the patient's anatomy; f removing the apparatus of the first aspect from the patient;
g. sensing the orientation of a surgical device with an orientation sensor;
h. monitoring the orientation of the surgical device relative to the monitored orientation of the patient's anatomy and guiding the surgical device to an optimal orientation relative to the monitored patient's anatomy.

Any of the features described herein can be combined in any combination with any one or more of the other features described herein within the scope of the invention.

### BRIEF DESCRIPTION OF DRAWINGS

Examples of the invention will now be described by way of example with reference to the accompanying Figures, in which:

FIG. 1 is a perspective view of an apparatus for aligning a sensor relative to at least two anatomical reference points of a patient's anatomy according to an example of the present invention;

FIG. 2 is a top view of a patient sensor support body according to an example of the present invention;

FIG. 3 is a side view of the patient sensor support body shown in FIG. 2;

FIG. 4 is a cross sectional view of the patient sensor support body of FIG. 3 through line A-A;

FIG. 5 is a top view of a patient sensor support according to a second example of the present invention, including two fasteners;

FIG. 6 is a partial bottom view of the patient sensor support shown in FIG. 5, illustrating a clamp in an open position;

FIG. 7 is an elevation view of an orientation sensor according to one example of the present invention;

FIG. 8 is a plan view of the orientation sensor shown in FIG. 7;

FIG. 9 is an elevation view of a patient sensor/apparatus sensor according to one example of the present invention;

FIG. 10 is a plan view of the patient sensor/apparatus sensor shown in FIG. 9;

FIG. 11 is a perspective view of an orientation sensor mounted to a surgical device (a placement device for placing an acetabular cup) according to one example of the present invention;

FIG. 12 is a perspective view of an orientation sensor mounted to a surgical device (an acetabular reamer) according to another example of the present invention;

FIG. 13 is an exemplary output from a monitor, showing how deviations in the patient position on the operating table and anatomical deviations in the patient may be inputted to adjust the optimal orientation angle; and

FIG. 14 is an exemplary output from a monitor, showing the pitch, roll and yaw of the surgical device relative to the optimal pitch, roll and yaw of the surgical device relative to the patient's anatomy.

Preferred features, embodiments and variations of the invention may be discerned from the following Description which provides sufficient information for those skilled in the art to perform the invention. The following Description is not to be regarded as limiting the scope of the preceding Summary of the Invention in any way.

### DESCRIPTION OF EMBODIMENTS

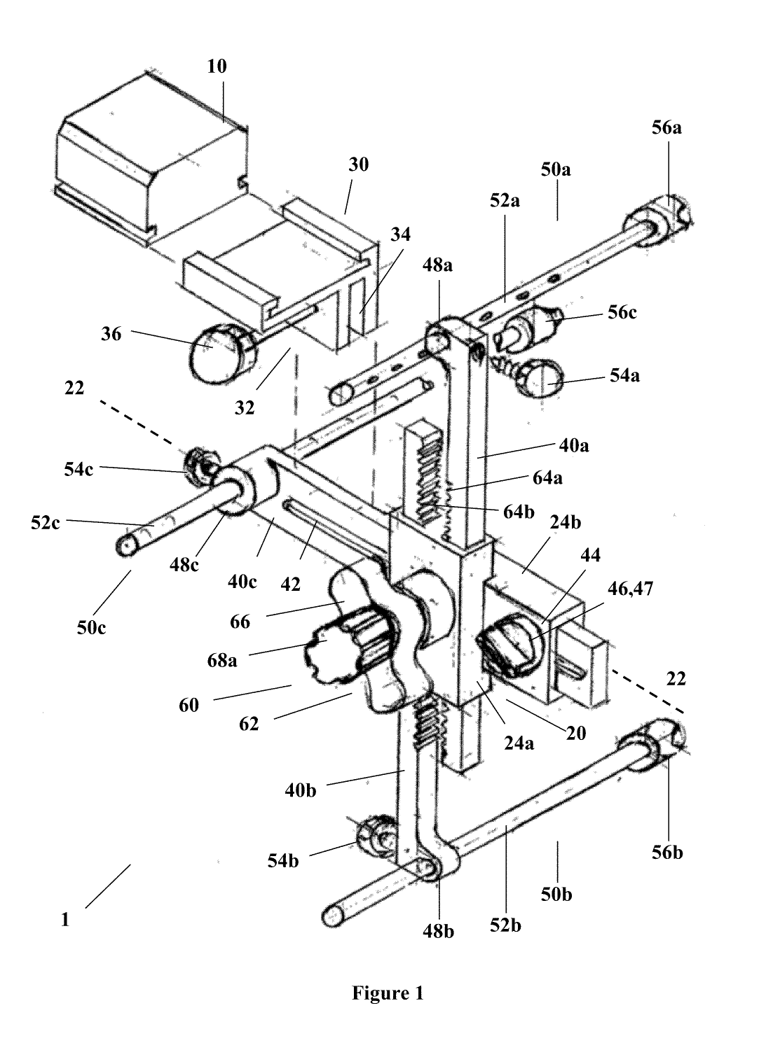

Embodiments and features of the present invention are illustrated with reference to FIGS. 1 to 14. In the figures, like numbers refer to like features.

FIG. 1 illustrates an apparatus 1 for aligning a sensor 10 relative to at least two anatomical reference points of a patient's anatomy. The apparatus 1 includes a body 20 having a central axis 22, a sensor mount 30, and at least two arms 40 extending from the body 20, wherein two of said at least two arms (40a, 40b) are simultaneously and equidistantly moveable relative to the central axis 22. The apparatus 1 also includes at least two aligners 50 connected to the at least two arms 40 for aligning with said at least two anatomical reference points.

The apparatus 1 includes an arm mechanism 60 for simultaneously and equidistantly moving the arms 40a, 40b relative to the central axis 22. The arm mechanism 60 operates manually and is geared. The arm mechanism 60 is a rack and pinion mechanism. Therefore, the arm mechanism 60 includes a pinion 62 and the two arms 40a, 40b each include a rack 64a, 64b (in the form of teeth on the two arms 40a, 40b). Rotation of the pinion 62 results in simultaneous movement of the two arms 40a, 40b towards and away from the central axis 22. The pinion 62 includes a handle 66 for actuating the pinion 62. The arm mechanism 60 also includes a brake 68 to prevent movement of the two arms 40a, 40b. The brake 68 includes a brake pad (positioned between the handle 66 and the body 20—not shown in FIG. 1) and a knob 68a. Rotation of the knob 68a drives the pinion handle 66 into the brake pad, preventing the handle 66 from turning to actuate the pinion 62.

The apparatus 1 includes three arms 40a, 40b, 40c (collectively “40”). The three arms 40 generally extend relative to each other in a “T”-shape. Each arm 40 is substantially straight.

The body 20 includes two sleeves 24a, 24b (collectively “24”). Arms 40a, 40b are supported by and extend through sleeve 24a. Arm 40c is supported by and extends through sleeve 24b. Each sleeve 24 is substantially rectangular in shape. Sleeve 24a is oriented substantially perpendicularly to sleeve 24b.

Arm 40c extends along the central axis 22. Arm 40c includes a longitudinally extending slot 42. The apparatus 1 further includes a lock 44 for the third arm 40c. The lock 44 includes a fastener 46 having a handle 47. The fastener 46 traverses two opposed walls of sleeve 24b through slot 42. Rotation of fastener 46 draws the walls of the sleeve 24b together, thereby locking arm 40c in position.

Sensor mount 30 may be releasably attached to the third arm 40c. Sensor mount includes a clamp 32. Clamp 32 includes a channel 34 and a fastener 36 in the form of a screw, in which the fastener 36 traverses the two opposed walls of the channel 34 and passes through the slot 42 of third arm 40c. Rotation of the fastener 36 draws the walls of the channel 34 together to clamp the sensor mount 30 on arm 40c.

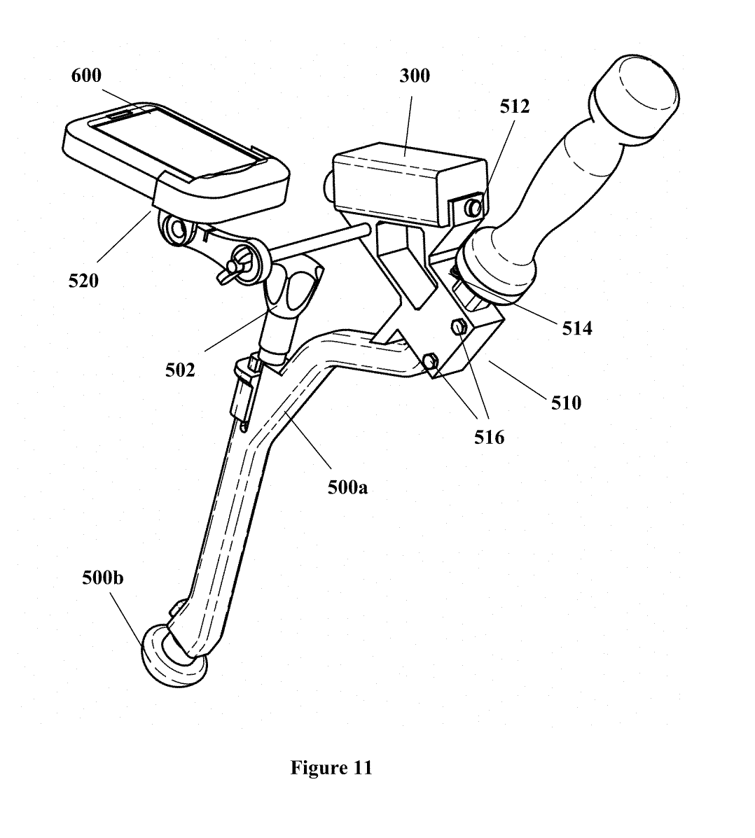

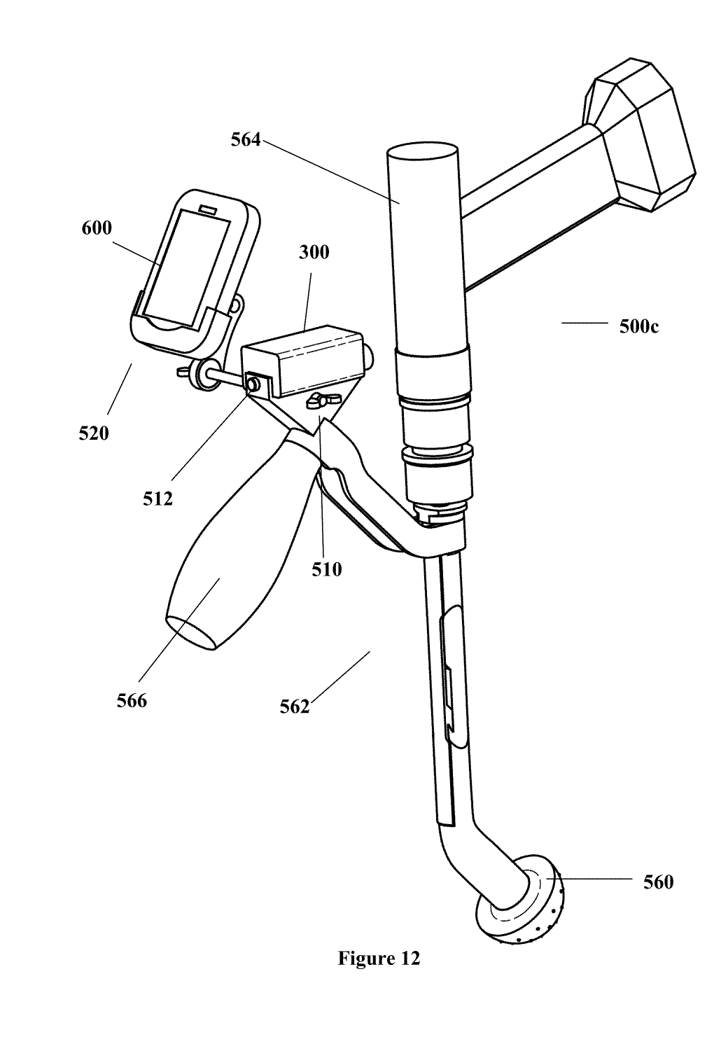

The apparatus also includes apparatus sensor 10. Apparatus sensor 10 is in slideably engageable with the sensor mount. An exemplary apparatus/patient sensor 100 is illustrated in FIGS. 9 and 10.

The apparatus/patient sensor 100 includes a power supply 102 (in the form of one or two batteries), a housing 104, and a sensor 106. The battery (or batteries) is especially sterilisable, and most especially is a lithium ion battery (e.g. 3.6V). The battery may be rechargeable or non-rechargeable. The sensor 106 is able to measure orientation in three axes (pitch, roll and yaw), and contains a wireless data transmission (such as Blue Tooth™ or Wi-Fi connection). The sensor 106 may include pitch and roll sensors with 2.4 GHz wireless transmission, and a yaw sensor with an ultra-sensitive magnetometer. The sensor 106 may be non-magnetisable and be able to be quickly and securely positioned in the apparatus/patient sensor 100. The apparatus/patient sensor 100 may also include an indicator, such as a light, for indicating the power remaining in the power supply 102. The apparatus/patient sensor 100 may be a 3 axis self-powered Wi-Fi transmitter.

Referring to FIG. 1, apparatus 1 also includes an aligner 50a, 50b, 50c (collectively “50”) connected to each arm 40. The aligners 50 are oriented perpendicularly to a substantially common plane within which the arms 40 lie. The aligners 50 are all moveable in a direction perpendicular to the direction of movement for each arm 40.

Each arm 40 terminates at a sleeve 48a, 48b, 48c (collectively “48”), and each aligner 50 is moveable within said sleeve 48. Each aligner 50 includes a rod 52a, 52b, 52c (collectively “52”) moveable within each sleeve 48. Each rod 52 includes a plurality of detents or apertures engageable with fasteners 54a, 54b, 54c (collectively “54”) (e.g. screws) mounted to each arm sleeve 48 for preventing movement of the aligner 50. Each aligner 50 is the same length.

Each aligner 50 includes an end 56a, 56b, 56c (collectively “56”) for alignment with an anatomical reference point. Each end 56 is blunt and bulbous, but has a contoured shape to conform to the anatomical reference point. Ends 56a, 56b are designed to be aligned with the two anterior superior iliac spines and may include a groove, and end 56c is designed to be aligned with at least one pubic crest and may include a central ridge (i.e. the patient's anatomy is the pelvis). The pad at the terminus of each end 56 is of 10-20 cm in diameter.

The aligner ends are made from heat resistant nylon, the aligner rods are made from titanium, and where allowing, the remainder of apparatus 1 is made from non-magnetisable stainless steel grade 316.

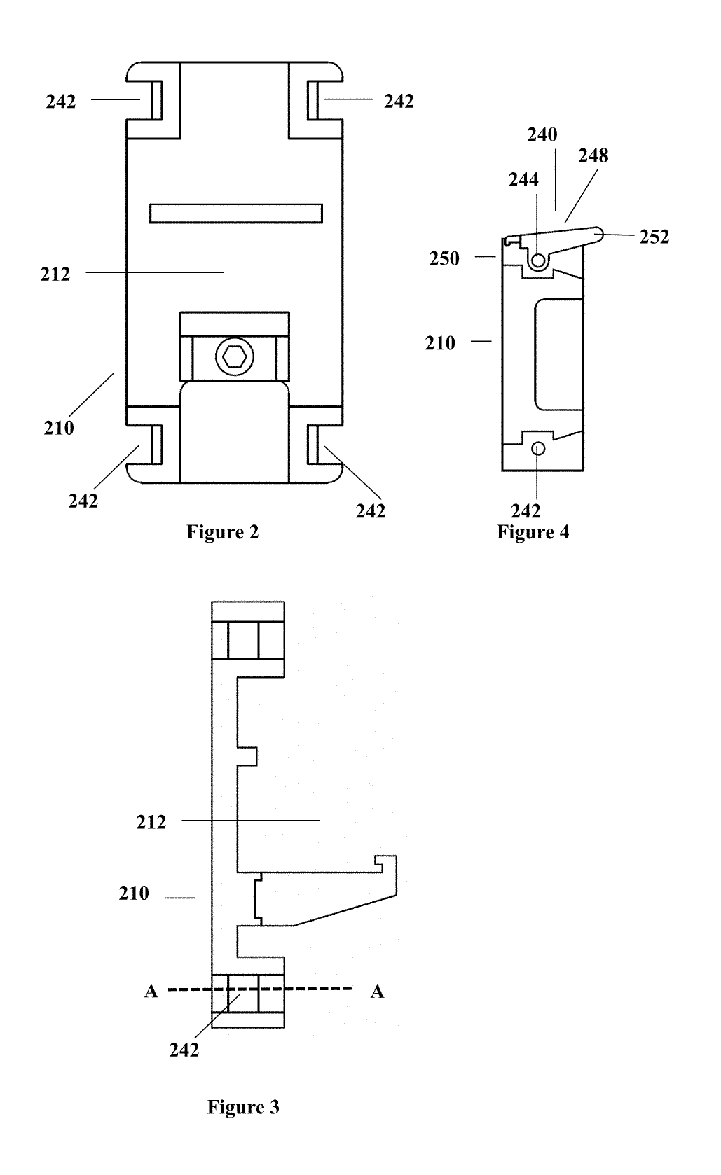

The apparatus 1 may form part of a surgical system. The surgical system may further include a patient sensor 100 and patient sensor support 200. The patient sensor 100 is mountable relative to the patient's anatomy and is for sensing changes in the orientation of the patient's anatomy. The patient sensor supports 200 illustrated in FIGS. 2-6 are intended to be mounted relative to the sacrum, and the patient sensor 100 is for sensing changes in the orientation of the patient's pelvis. The patient sensor support 200 is substantially rigid.

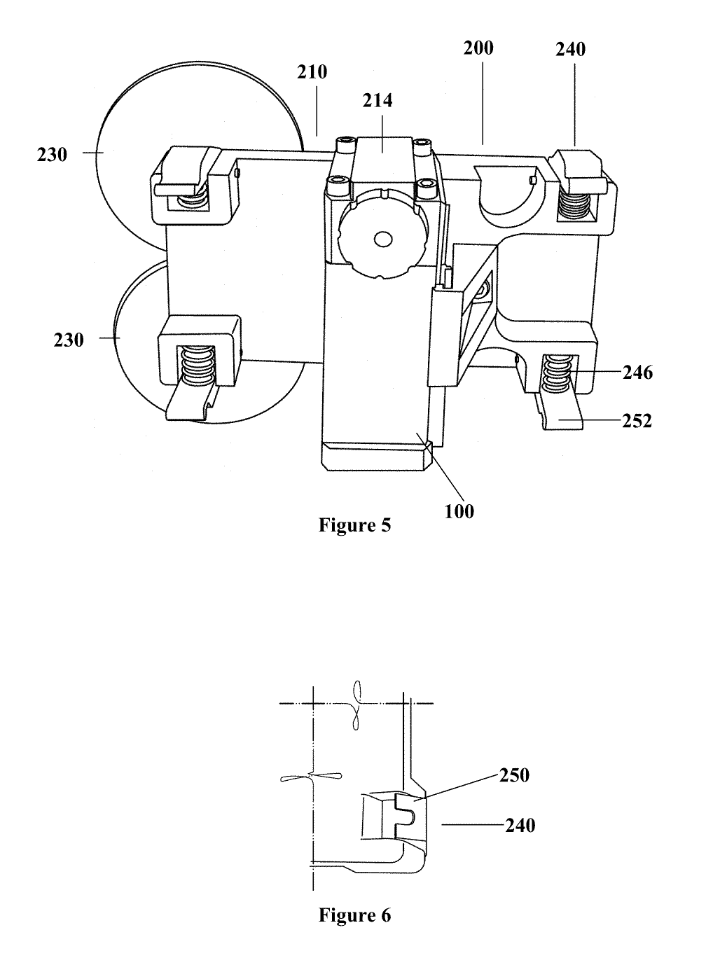

The patient sensor support 200 includes a body 210 which is of substantially rectangular shape. The body 210 includes a patient sensor mount 212 for mounting the patient sensor 100. The patient sensor 100 is slideably engageable with the patient sensor mount 212. Patient sensor mount 212 may include a clamp 214 to secure the patient sensor, as illustrated in FIG. 5.

The patient sensor support 200 includes at least one fastener 230. The patient sensor supports 200 illustrated in FIGS. 2-6 are intended to include four fasteners 230, one at each corner. The fasteners 230 are in the form of adhesive tabs (such as those used in ECG) which have an adhesive surface for adhering to the patient's skin. The adhesive tabs also have a projection (which may be substantially cylindrically shaped) opposite the adhesive surface.

The patient sensor support 200 also includes four clamps 240 (see FIGS. 4 to 6; the space where the clamps 240 are attached to the body 210 is marked in FIGS. 2-4 at 242). The clamps 240 are pivotally mounted to the body 210 at 244 (see FIG. 4). Each clamp 240 includes a biasing member 246 (see FIG. 5) in the form of a spring to bias the clamp to a closed position. The clamp includes a lever 248. The lever 248 has a jaw 250 at one end and an actuator 252 at an opposite end. The actuator 252 is depressed by an operator to move the clamp 240 to an open position.

The patient sensor support body may be made of any suitable material, such as polycarbonate. The patient sensor support is sterilisable.

The surgical system is configured to monitor the orientation of the patient's anatomy by combining the initial orientation sensed by the apparatus sensor 10 with the changes in orientation sensed by the patient sensor 100. This may be achieved by pairing the sensors 10, 100, or by transmitting the data to a monitor for processing.

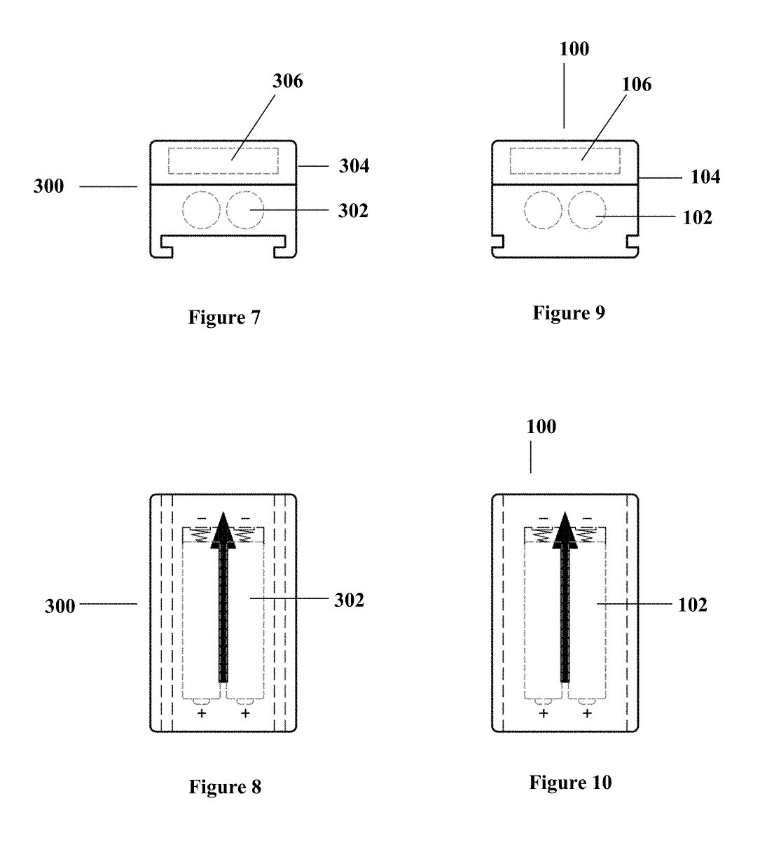

The surgical system may also include an orientation sensor 300 (see FIGS. 7, 8, 11 and 12). The orientation sensor 300 may include power supply 302 (in the form of battery), a housing 304, and a sensor 306. Features of the orientation sensor 300 may be as described above for the patient/apparatus sensor 100.

As shown in FIGS. 8 and 10, the sensors 100, 300 have an arrow pointing in the sensor pairing direction which will, when attached to the apparatus sensor mount 30, the patient sensor support 200 or the orientation sensor mount 510, be the direction towards the patient's head when in use.

The orientation sensor 300 may be mounted to a surgical device 500. An exemplary surgical device includes a placement device for placing a prosthetic component together with the prosthetic component (such as an acetabular cup inserter 500a and acetabular cup 500b, as shown in FIG. 11). A further exemplary surgical device includes a surgical implement such as a reamer (such as the acetabular reamer 500c shown in FIG. 12). In either case, the orientation sensor 300 may be mounted to the surgical device 500 by way of an orientation sensor mount 510.

The acetabular cup inserter 500a illustrated in FIG. 11 is a curved acetabular cup inserter (the knob 502 is for releasably coupling the acetabular cup 500b). An exemplary acetabular cup inserter 500a is manufactured by DePuy (Catalogue Number 920010029). The orientation sensor mount 510 includes a docking port 512 into which the orientation sensor 300 is screwed (an alternative arrangement in which orientation sensor 300 is slideably engaged with docking port 512 may also be used). The mount 510 also includes a bracket 520 for holding a monitor 600 (in the form of a tablet (or an iPad®) or a smartphone (such as an iPhone®) or the like). In use, the tablet or smartphone may be encased within a sterilisable housing which is held in, or forms part of the bracket 520. Alternatively, the mount 510 does not include bracket 520. Instead, the system further includes a stand for monitor 600 which is independent to the mount 510. The stand may be positionable on the floor of the operating theatre and be located in visual proximity to the surgeon. A suitable stand may be a music stand for an iPad or the like.

The mount 510 also includes a shock absorber 514. The shock absorber 514 may be biased against the handle of the placement device 500a, and may be assembled with an impact shaft (spool) (preferably titanium), a coil spring (preferably stainless steel), and a pneumatic compression chamber. A collar on the titanium spool may abut the spring, and the end of the titanium spool may extend partway into the compression chamber. However, the pneumatic compression chamber is optional. Other types of shock absorbers may be used, such as a two-direction shock absorber.

The mount 510 may include a clamp 516 for clamping the mount 510 to the placement device 500a. The clamp 516 illustrated in FIG. 11 includes a fastener such as bolts and lock nuts. The mount 510 may be made from a sterilisable material, and the body of the mount 510 may be especially made of stainless steel (such as grade 316). The mount 510 is especially made of a non-magnetisable material.

An acetabular reamer 500c includes a cutting blade 560, an acetabular reamer driver 562, and a motor 564. The acetabular reamer 500c has a slot next to a handle 566 (the handle 566 forms part of the acetabular reamer driver 562), to which the orientation sensor mount 510 may be clamped. An exemplary acetabular reamer driver is the DePuy angled reamer driver catalogue number 920010031.

However, in the acetabular reamer 500c illustrated in FIG. 12 the acetabular reamer driver 562 is integrally formed with orientation sensor mount 510. The mount 510 includes a docking port 512 into which the orientation sensor 300 may be screwed (an alternative arrangement in which orientation sensor 300 is slideably engaged with docking port 512 may also be used), and bracket 520 for holding a monitor 600 in the form of a tablet (or an iPad®) or smartphone (such as an iPhone®) or the like. In use, the tablet or smartphone may be encased within a sterilisable housing which is held in, or forms part of the bracket 520. Alternatively, mount 510 does not include bracket 520. Instead, the system further includes a stand for monitor 600 which is independent to the mount 510. The stand may be positionable on the floor of the operating theatre and be located in visual proximity to the surgeon. A suitable stand may be a music stand for an iPad or the like.

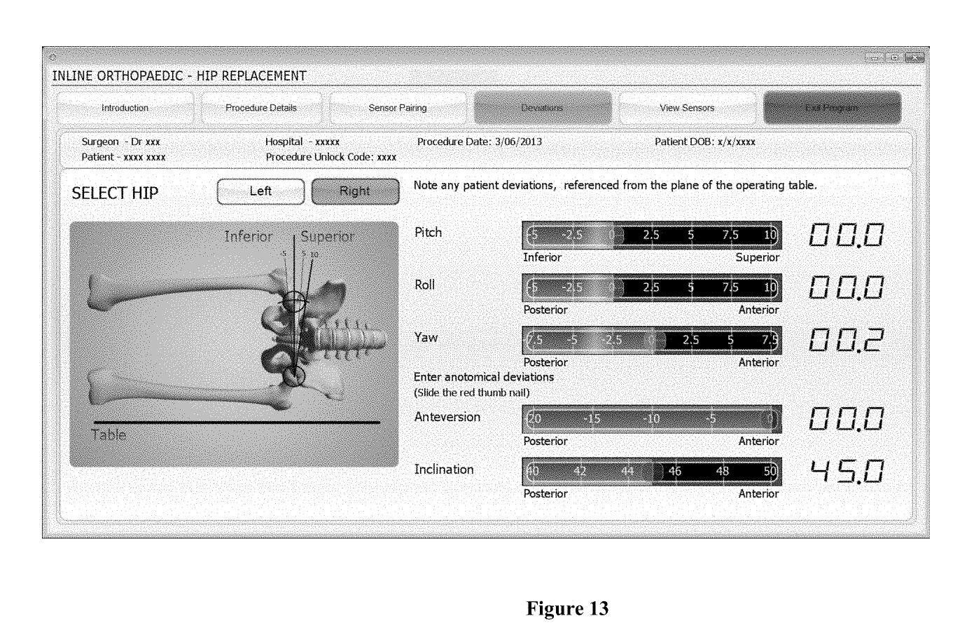

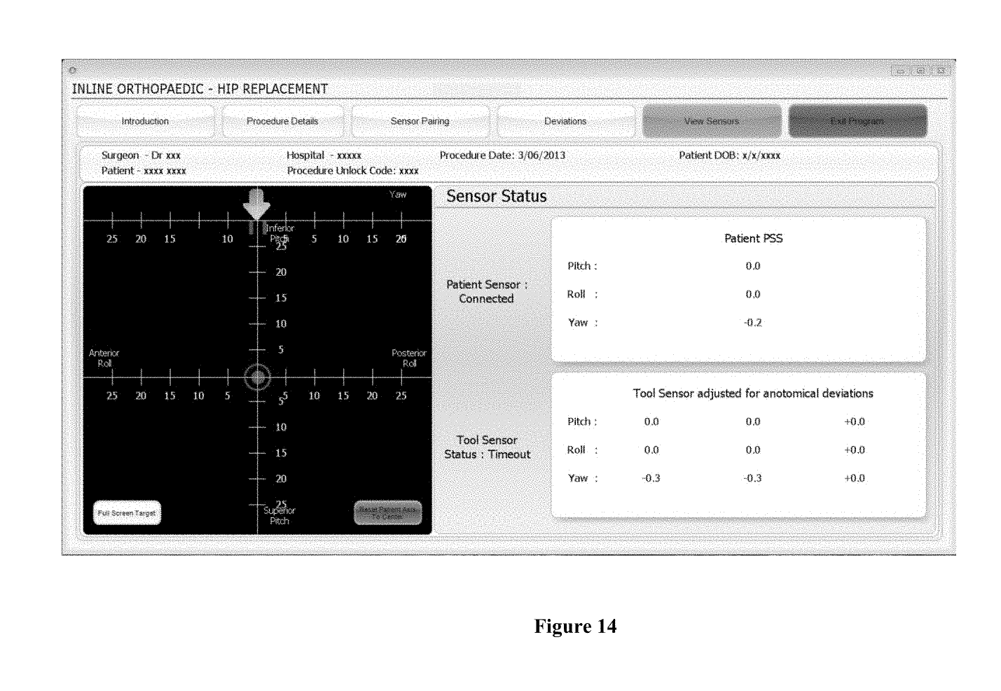

The surgical system may be used according to the following steps:
1. Place the patient on the operating table, preferably lying on their side.
2. Start software using monitor 600 (e.g. computer (including a tablet or iPad®)).
3. Enter details of the procedure, including the surgeon's name, the patient's name, the hospital name, the date of birth of the patient and the date of the procedure.
4. Enter any offsets for inclination and anteversion (or pitch, roll and yaw) in view of the patient's anatomy (for example due to natural variations in the patient's anatomy). See FIG. 13. This information may be obtained through a pre-operative scan such as an X-ray.
5. Place apparatus, patient and orientation sensors in a tray so that all three sensors are generally uniformly aligned. Pair (calibrate) the sensors, such that the pitch, roll and yaw for all sensors are substantially zero.
6. Place patient sensor support 200 over patient's sacrum. Insert patient sensor 100 into patient sensor mount 212.
7. Move arms 40 of alignment apparatus 1 so that aligners 50a, 50b align with the patient's anterior superior iliac spines, and so that aligner 50c aligns with the patient's at least one pubic crest. Move aligners 50 within arm sleeves 48 so that apparatus sensor mount 30 is positioned close to patient's body. Set all aligners 50 at the same height relative to arms 40. Insert apparatus sensor 10 into mount 30.
8. When the apparatus 1 is in the correct position, pair (or lock) patient and apparatus sensors 10,100 so that the system monitors the orientation of the patient's pelvis. This information is used to define the patient's coronal (and possibly also sagittal) planes. Data from the apparatus sensor and patient sensor provides an initial orientation of the patient's pelvis and also changes in the orientation of the patient's pelvis, which together provides a monitored orientation of the pelvis. Using this data and the data previously obtained, the optimal orientation of the surgical device relative to the patient's pelvis may be calculated.
9. Remove apparatus 1 from patient.
10. Assemble and make ready an appropriate pelvic support brace and set the patient ready for covering;
11. Insert orientation sensor into orientation sensor mount on surgical device.
12. View on the monitor 600 the orientation of the surgical device relative to the patient's anatomy, and also the difference between the present orientation of the surgical device and the optimal orientation. See FIG. 14.
13. Perform the acetabular reaming or acetabular cup placement operation.
14. A record of the output of the surgical system during the operation may be stored on a memory card for future reference.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

1. A surgical system for monitoring the orientation of a patient's anatomy, wherein the system includes:
a. an apparatus for aligning an apparatus sensor relative to at least two anatomical reference points of a patient's anatomy to thereby sense an initial orientation of the patient's anatomy, wherein the apparatus includes:
i. a body;
ii. the apparatus sensor positioned relative to the body;
iii. at least two arms extending from the body; and
iv. at least two aligners connected to the at least two arms for aligning the apparatus with said at least two anatomical reference points; and
b. a patient sensor mountable relative to the patient's anatomy by way of a patient sensor support, wherein the patient sensor is for sensing changes in the orientation of the patient's anatomy, and wherein the patient sensor support includes a fastener for mounting the patient sensor support to the patient's skin;
wherein the surgical system is configured to monitor the orientation of the patient's anatomy by combining the initial orientation sensed by the apparatus sensor with the changes in orientation sensed by the patient sensor.

2. The surgical system of claim 1, wherein the fastener includes an adhesive for adhering the patient sensor support to the patient's skin.

3. The surgical system of claim 1, wherein the apparatus sensor and/or the patient sensor comprise at least one sensor for sensing the orientation of the patient's anatomy, wherein the at least one sensor is selected from one or more of a gyroscope, a magnetometer, an accelerometer, an inclinometer and an inertial sensor.

4. The surgical system of claim 1, wherein the body of the apparatus has a central axis, and two of said at least two arms are simultaneously and equidistantly moveable relative to the central axis.

5. The surgical system of claim 4, wherein the apparatus further includes a rack and pinion mechanism for simultaneously and equidistantly moving said two of said at least two arms relative to the central axis.

6. The surgical system of claim 4, wherein said at least two arms extend in a substantially common plane.

7. The surgical system of claim 4, wherein said two of said at least two arms extend on opposite sides of the central axis.

8. The surgical system of claim 4, wherein the apparatus includes a third arm extending from the body, and a third aligner connected to the third arm for aligning with a third anatomical reference point, wherein said third arm is moveable relative to the body.

9. The surgical system of claim 8, wherein the third arm extends along the central axis.

10. The surgical system of claim 1, wherein said at least two aligners extend substantially parallel to each other.

11. The surgical system of claim 1, wherein the patient's anatomy is the pelvis.

12. The surgical system of claim 11, wherein the at least two anatomical reference points are selected from the group consisting of: one or two anterior superior iliac spines, one or two posterior superior iliac spines, at least one pubic crest and the sacrum.

13. A surgical system for guiding a surgical device to an optimal orientation relative to a patient's anatomy, wherein the surgical system includes:
a. the surgical system for monitoring the orientation of a patient's anatomy of claim 1;
b. an orientation sensor for sensing the orientation of the surgical device; and
c. a monitor for monitoring the orientation of the surgical device relative to the monitored orientation of the patient's anatomy, and for guiding the surgical device to an optimal orientation relative to the monitored patient's anatomy.

14. The surgical system of claim 13, wherein the surgical device is a surgical implement or includes a prosthetic component.

15. The surgical system of claim 13, wherein the patient's anatomy is the pelvis.

16. A method of monitoring the orientation of a patient's anatomy using the surgical system of claim 1, wherein the method includes the steps of:
a. using the apparatus to align the apparatus sensor relative to the at least two anatomical reference points of the patient's anatomy;
b. sensing the orientation of the patient's anatomy with the apparatus sensor, wherein the apparatus sensor is for sensing an initial orientation of the patient's anatomy;
c. mounting the patient sensor relative to the patient's anatomy by mounting the patient sensor support to the patient's skin;
d. sensing the orientation of the patient's anatomy with the patient sensor, wherein the patient sensor is for sensing changes in the orientation of the patient's anatomy; and
e. pairing the apparatus sensor with the patient sensor, to thereby monitor the orientation of the patient's anatomy.

17. The method according to claim 16, which is a method of guiding a surgical device to an optimal orientation relative to a patient's anatomy, wherein the method further includes the steps of:
f. removing the apparatus from the patient;
g. sensing the orientation of a surgical device with an orientation sensor; and
h. monitoring the orientation of the surgical device relative to the monitored orientation of the patient's anatomy and guiding the surgical device to an optimal orientation relative to the monitored patient's anatomy.
